(19) United States
(12) Patent Application Publication
(10) Pub. No.: US 2007/0053955 A1
(43) Pub. Date: Mar. 8, 2007

(54) Compositions and methods for improving cellular survival and reducing apoptosis after ischemic episode in the brain

(75) Inventors: Brian Larson, Washington, MO (US); Yuanlong Pan, Chesterfield, MO (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: NESTEC S.A., Vevey (CH)

(21) Appl. No.: 11/510,358

(22) Filed: Aug. 25, 2006

Related U.S. Application Data
(60) Provisional application No. 60/711,547, filed on Aug. 26, 2005.
(60) Provisional application No. 60/711,549, filed on Aug. 26, 2005.

(51) Int. Cl. A61K 31/202; A61K 31/198

(52) U.S. Cl. 424/439; 514/560; 514/565; 424/442

(57) ABSTRACT

Compositions and methods for enhancing vascular integrity in animals are disclosed. The compositions and methods, which utilize long chain polyunsaturated fatty acids and nitric oxide releasing compounds are also effective for reducing ischemia-induced brain injury in an animal.

### CROSS REFERENCE TO RELATED APPLICATIONS

This claims benefit of U.S. Provisional Patent Application Nos. 60/711,547 and 60/711,549, filed Aug. 26, 2005, the entire contents of both of which are incorporated herein by reference.

### FIELD OF THE INVENTION

The present invention relates to mammalian nutrition and effects thereof on the enhancing vascular integrity in the brain. In particular, the present invention relates to utilizing combinations of nitric oxide-releasing compounds and long chain polyunsaturated fatty acids, administered as part of a regular diet, to improve vascular integrity and reduce ischemic injury in the brain in the event of a brain ischemia.

### BACKGROUND OF THE INVENTION

Various publications, including patents, published applications, technical articles and scholarly articles are cited throughout the specification. Each of these cited publications is incorporated by reference herein, in its entirety.

Hypoxic ischemic brain injury occurs as the result of diminished oxygen supply to the brain due to a transient or permanent reduction or stoppage of blood flow to the brain. Ischemia of the brain results in a stroke, with subsequent apoptosis and necrosis of brain tissue leading to an infarction. Similar to cardiovascular ischemia, brain ischemia can be caused by various factors such as blood clots, thrombosis, embolism, blockage by atherosclerotic plaques, or other obstructions in the vasculature. Hypercholesterolemia, hypertension, diabetes, and obesity, among other factors, have been identified as risk factors for ischemic strokes. Ischemic strokes are a leading cause of death of human beings worldwide, and also affect other animals, including companion animals.

During ischemic stroke, biochemical reactions occur in the vasculature that may lead to edema, hemorrhagic transformation, and a further compromise in neurological tissue. As such, treatment and protection of the vasculature has been identified as a potential avenue to explore in acute ischemic stroke in order to develop new therapies. Moreover, vascular protection has implications for reducing tissue damage that occurs as a result of an ischemic episode.

The vascular endothelium has been determined to serve various regulatory functions, including modulation of vascular tone, inflammation, and homeostasis by maintaining a non-adhesive, anti-thrombotic surface. (Boak, L., et al. Cur. Vasc. Pharmacol. (2004) 2:45-52). Vascular homeostasis is, in part, a function of the vascular smooth muscle contraction and relaxation, which is mediated by a variety of factors, including the free radical, nitric oxide (NO), which is a strong relaxant of vascular smooth muscle.

NO is produced by three different NO synthase (NOS) enzymes, neuronal NOS (nNOS), which is primarily localized in nervous tissue and generates NO for neurotransmission; inducible NOS (iNOS), which is found primarily in macrophages and may respond to proinflammatory mediators; and endothelial NOS (eNOS), which is produced by endothelial cells such as those found in the vascular endothelium. (Michel, T., et al. J. Clin. Invest. (1997) 100: 2146-2152, Moncada, S., et al. Pharmacol. Rev. (1991) 43:109-142, and, Nathan, C. (1992) FASEB J 6: 3051-3064). NO released by eNOS plays a major role in vasodilation, smooth muscle proliferation, and regulation of arterial blood pressure. It also has anticoagulant and anti-inflammatory effects via inhibition of adhesion and aggregation of platelets and leukocytes. (Gewaltig, M. T., et al., Cardiovasc. Res. (2002) 55:250-260, De Graaf, J. C. et al. Circulation (1992) 85:2284-2290, Freedman, J. E., et al., Circ. Res. (1999) 84:1416-1421, Furchgott, R. F., et al., FASEB J (1989) 3:2007-2018, Gaboury, J., et al., Am J Physiol. Heart. Circ. Physiol. (1993) 265: H862-H867, and, Kubes, P., et al., Proc. Natl. Acad. Sci. USA (1991) 88: 4651-4655). Decrease in NO production during prolonged ischemia has been associated with endothelial injury (Laude, L., et al., Braz. J. Med. Biol. Res. (2001) 34:1-7).

The NOS enzymes oxidize L-arginine to citrulline, resulting in the formation of NO as a byproduct. Thus, it has been suggested that supplementation with NO precursors such as L-arginine, can improve vascular health, and may facilitate repair of vascular disease states. Indeed, dietary arginine supplementation has been shown to induce restoration of vasodilation and improve coronary circulation in animal models and human patients with hypercholesterolemia, as well as enhance overall endothelial function in patients with coronary artery disease. (Boak, L., et al. 2004). In addition, L-arginine supplementation has also been shown to increase NO levels and attenuate free O₂ radical-mediated myocardial injury in human patients. (Kiziltepe, U., et al. Int. J. Cardiol. (2004) 97:93-100). More recent studies have shown that arginine supplementation facilitates endothelial repair in the intestine of rats following induced intestinal ischemia-reperfusion. (Sukhotnik, I., et al., Pediatr. Surg. Int. (2005) 3:191-196). Thus, administration of NO precursors such as L-arginine have implications for endothelial cell activation and protection, especially with respect to vascular endothelial cells.

Fatty acids have also been demonstrated to modulate endothelial cell activation. Administration of the long chain polyunsaturated fatty acids (LCPUFA) was found to inhibit adhesion of lymphocytes to vascular endothelial cells. (Khalfoun, B., et al., Transplantation (1996) 62:1649-1657). Subsequent investigations determined that administration of LCPUFA reduces endothelial cell expression of adhesion molecules and cytokines in response to stimulation, suggesting that LCPUFA have anti-atherogenic and anti-inflammatory properties. (De Caterina, R., et al., Am. J. Clin. Nutr. (2000) 71(suppl)213S-223S). Mounting evidence indicates that regular consumption of LCPUFA, in particular, n-3 fatty acids, protects the cardiovascular system, reduces atherosclerotic plaque formation, and reduces the risk of mortality from cardiovascular disease, particularly following a myocardial infarction. (Calder, P. C., Clin. Sci. (Lond). 2004 107: 1-11). In contrast, there has been little investigation of the effect of dietary LCPUFA on the vasculature and tissue of the brain, particularly with respect to an ischemic episode in the brain.

### SUMMARY OF THE INVENTION

One aspect of the present invention features a composition comprising one or more long chain polyunsaturated fatty acids (LCPUFA) and one or more nitric oxide releasing compounds (NORC) in an amount effective for the enhancement of vascular integrity in the brain of an animal. The enhanced vascular integrity of the brain serves to reduce ischemic injury in the brain in the event of an ischemic episode, and may also exert a protective effect against the initial occurrence of such ischemic events.

In certain embodiments, the composition is a pet food composition, or a dietary supplement. In other embodiments the composition is a food product or supplement formulated for human consumption. In various embodiments, the LCPUFA include at least one of arachidonic acid, eicosapentaenoic acid, docosapentaenoic acid, or docosahexaenoic acid, and the NORC include at least one of L-arginine or derivatives thereof. The compositions may comprise additional ingredients. For example, one or more antioxidants may be included.

In certain embodiments, the compositions are formulated for companion animals, such as a dog or a cat. In other embodiments, the compositions are formulated for human consumption.

Another aspect of the invention features a method for reducing ischemia-induced brain injury in an animal, comprising administering to the animal on a regular basis a composition comprising one or more LCPUFA and one or more NORC, as described above, in an amount effective to reduce ischemia-induced brain injury in the event of an ischemic episode in the brain of the animal. In certain embodiments, the method is applied to a companion animal, such as a dog or a cat. In other embodiments, the method is applied to humans.

Other features and advantages of the invention will become apparent by reference to the drawings, detailed description and examples that follow.

### BRIEF DESCRIPTION OF THE DRAWINGS

FIG. 1. Bar graph showing effect of control and test diets on ischemic lesion volume in all rats subjected to transient cerebral ischemia. Compositions of control and test diets are described in Example 1 (*p<0.01 vs control).

FIG. 2. Bar graph showing effect of control and test diets on ischemic lesion volume in rats exhibiting lesions after transient cerebral ischemia. Compositions of control and test diets are described in Example 1 (*p<0.01 vs control).

FIG. 3. Bar graph showing effect of control and test diets on cerebral ischemia-induced apoptosis in rats. Compositions of control and test diets are described in Example 1 (*p<0.001 vs control).

FIG. 4. Graph showing correlation between ischemic lesion volume (X axis) and percentage of apoptotic cells as defined by TUNEL staining (Y axis) in rats subjected to transient cerebral ischemia. Compositions of control and test diets are described in Example 1.

### DETAILED DESCRIPTION OF ILLUSTRATIVE EMBODIMENTS

Definitions

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The following abbreviations may be used in the specification and examples: AA, arachidonic acid; ALA, alpha-linolenic acid; ANOVA, analysis of variance; DHA, docosahexaenoic acid; DM, dry matter; DPA, docosapentaenoic acid; EPA, eicosapentaenoic acid; LA, linoleic acid; LCPUFA, long chain polyunsaturated fatty acids (as used herein LCPUFA refers to one or more such fatty acids); NO, nitric oxide; NORC, nitric oxide releasing compound or compounds; NOS, nitric oxide synthase; eNOS, endothelial Notric oxide synthase; iNOS, inducible nitric oxide synthase; nNOS, neuronal nitric oxide synthase; L-Arg, L-arginine.

“Effective amount” refers to an amount of a compound, material, or composition, as described herein that achieves, or results, in a particular biological response, e.g. a desired biological response. Such results include, but are not limited to, enhancing vascular integrity or reducing ischemic brain injury. Such activity may be achieved, for example, by administering the compositions of the present invention to the animal in an effective amount.

The term “vascular integrity” refers to the overall health of the blood vessels in an animal, including, without limitation, healthy vascular tone and structure, healthy vascular growth, healthy constriction and dilation, diminished permeability, diminished proliferation, diminished inflammation, diminished cell and/or platelet adhesion, maintenance of normal blood pressure and blood flow, and the like. “Enhanced vascular integrity” refers to any improvement in the overall health of the blood vessels in an animal, as measured by any means suitable fro such purposes, such as are known in the art.

As used herein, “long chain polyunsaturated fatty acids” or “LCPUFA” refers to any one or more monocarboxylic acids having at least 20 carbon atoms and at least two double bonds. Non-limiting examples of LCPUFA include (n-6) fatty acids, such as arachidonic acid (AA), and (n-3) fatty acids, such as eicosapentaenoic acid (EPA), docosapentaenoic acid (DPA) and docosahexaenoic acid (DHA).

As used herein, “nitric oxide releasing compounds” or “NORC” refers to any compound or compounds that causes or can result in the release of nitric oxide in an animal. Preferred examples of such compounds include L-arginine, and analogs or derivatives thereof, such as arginine alpha-ketoglutarate, GEA 3175, sodium nitroprusside, glyceryl trinitrate, S-nitroso-N-acetyl-penicillamine, nitroglycerin, S-NO-glutathione, NO-conjugated non-steroidal anti-inflammatory drugs (e.g. NO-naproxen, NO-aspirin, NO-ibuprofen, NO-Diclofenac, NO-Flurbiprofen, and NO-Ketoprofen), NO-releasing compound-7, NO-releasing compound-5, NO-releasing compound-12, NO-releasing compound-18, diazeniumdiolates and derivatives thereof, diethylamine NONOate, and any organic or inorganic compound, biomolecule, or analog, homolog, conjugate, or derivative thereof that causes the release of nitric oxide, particularly “free” NO, in an animal.

“Ischemia” refers to any decrease or stoppage in the blood supply to any bodily organ, tissue, cell, or part caused by any constriction or obstruction of the vasculature, particularly where that decrease or stoppage leads to or would likely lead to ischemic damage to the bodily organ, tissue, cell, or part. “Ischemic episode” refers to any transient or permanent period of ischemia.

“Vasculature” refers to any network, or portion thereof, of blood vessels in the body of an animal, the blood vessels including, without limitation, arteries, veins, and capillaries.

“Vascular endothelial cells” or “vascular endothelium” refers to cells that comprise the layer of thin, flat cells that line the interior surface of blood vessels, forming an interface between circulating blood in the lumen and the rest of the vessel wall.

The present invention relates to any animal, preferably a mammal, more preferably companion animals, and most preferably humans. A “companion animal” is any domesticated animal, and includes, without limitation, cats, dogs, rabbits, guinea pigs, ferrets, hamsters, mice, gerbils, horses, cows, goats, sheep, donkeys, pigs, and the like.

As used herein, the term “pet food” or “pet food composition” means a composition that is intended for ingestion by an animal, and preferably by companion animals. A “complete and nutritionally balanced pet food,” is one that contains all known required nutrients in appropriate amounts and proportions based on recommendations of recognized authorities in the field of companion animal nutrition, and is therefore capable of serving as a sole source of dietary intake to maintain life or promote production, without the addition of supplemental nutritional sources. Nutritionally balanced pet food compositions are widely known and widely used in the art.

As used herein, a “dietary supplement” is a product that is intended to be ingested or administered, as a supplement to, or in addition, to the normal diet of an animal.

As used herein, a “food product formulated for human consumption” is any composition intended for ingestion by a human being.

As used herein, the term “food”, whether for human or nonhuman animals, includes compositions of any texture, consistency, moisture content, and the like, including both solid and nonsolid (for example, emulsions, suspensions, gels, and liquids) foods.

Description

The inventors have observed that supplemental arginine increased expression of multiple growth factors and anti-inflammatory proteins in a dose-dependent manner in dogs. Many such growth factors, including NGF, nIGF, and BDNF exert potent neuronal protective effects. (Mattson, M P et al., Neurobiol. Aging. (2002) 23:695-705, and Kruttgen, A. et al., Proc. Natl. Acad. Sci. USA (1998) 95:9614-9619). In further investigations of dogs, using natural sources of arginine, including herring meal (containing fish oil rich in LCPUFA), the inventors found an equal or better response than that observed using pure L-arginine. As described in detail in the examples herein, functional studies of transient cerebral ischemia in an ovariectomized rat model showed that a diet supplemented a combination of L-arginine, fish oil, antioxidants, and B-vitamins markedly reduced cerebral lesions and apoptosis, more so than diets supplemented with arginine or fish oil alone. Accordingly, various aspects of the present invention utilize these discoveries by providing dietary compositions and methods to improve vascular integrity of an animal and to reduce ischemic brain injury in the event of a brain ischemia in the animal.

Compositions

One aspect of the invention features compositions comprising one or more long chain polyunsaturated fatty acids (LCPUFA) and one or more nitric oxide releasing compounds (NORC) in an amount effective for the enhancement of vascular integrity in animals. The LCPUFA and NORC can be present in the composition as an ingredient or additive. In preferred embodiments of the composition, the LCPUFA comprise at least one of the (n-3) fatty acids such as ALA, EPA, DPA and DHA, and the NORC comprise at least one of L-Arg and derivatives thereof. The compositions enrich the blood plasma with LCPUFA and NORC in animals to which the composition is administered.

In one preferred embodiment, the compositions of the invention are pet food compositions. In one embodiment, the compositions include foods intended to supply necessary dietary requirements, as well as treats (e.g., biscuits), or other dietary supplements. Optionally, the pet food compositions comprise a dry composition (for example, kibble), semi-moist composition, wet composition, or any mixture thereof.

In another preferred embodiment, the compositions of the invention are food products formulated for human consumption. In various embodiments, they include foods and nutrients intended to supply necessary dietary requirements of a human being, as well as other human dietary supplements. In particular embodiments, the food products formulated for human consumption are complete and nutritionally balanced.

In another preferred embodiment, the composition is a dietary supplement, such as a water-based or other beverage, liquid concentrate, gel, gravy, powder, granule, paste, suspension, or emulsion. In certain embodiments, the composition can be provided as a chew, morsel, treat, snack, pellet, pill, capsule, tablet, caplet, concentrate or any other delivery form. In one embodiment the composition is provided in a formulation such as a yogurt. The dietary supplements can be specifically formulated for consumption by a particular animal, such as companion animal, or a human, or for a particular species of animal to fill a particular need.

In one embodiment, the dietary supplement comprises a high concentration of LCPUFA and NORC such that the supplement can be administered to the animal in small amounts, or in the alternative, can be diluted before administration to an animal. In various embodiments, the dietary supplement may require or simply permit admixing with water prior to administration to the animal.

The composition may be frozen, refrigerated, cryopreserved, or processed in other means to extend or preserve its useful life. The LCPUFA and NORC may be pre-blended with the other components, if any, of the composition to provide the beneficial amounts needed. The LCPUFA and NORC may also be coated onto a pet food composition, dietary supplement, or food product formulated for human consumption, or the like, or they may be added to the composition for example, as a powder or a mix, prior to offering it to, or administering it to the animal.

The compositions of the invention comprise LCPUFA and NORC in an amount effective to enhance vascular integrity in an animal to which the composition has been administered. For pet foods and food products formulated for human consumption, the amount of LCPUFA as a percentage of the composition is preferably in the range of about 0.1% to about 13% of the composition on a dry matter basis, although a greater percentage can be supplied. In various embodiments, the amount of LCPUFA is about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or more, e.g., 6%, 7%, 8%, 9%, 10%, 11%, 12%, 13% or more, of the composition on a dry matter basis. Preferably, the amount of NORC as a percentage of the composition is in the range of about 0.1% to about 12% of the composition on a dry matter basis, although a greater percentage can be supplied. In various embodiments, the amount of NORC is about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or more, e.g., 6%, 7%, 8%, 9%, 10%, 11%, 12% or more, of the composition on a dry matter basis. In specific embodiments, 2-2.5% LCPUFA and 2-2.5% NORC are utilized. In such embodiments, pure L-arginine is a preferred NORC compound. Dietary supplements may be formulated to contain several-fold higher concentrations of LCPUFA and NORC, to be amenable for administration to an animal in the form of a tablet, capsule, liquid concentrate, emulsion, suspension, gel, or other dosage form, or to be diluted before administrations, such as by dilution in water, or adding to a pet food (for example by spraying, or sprinkling thereon), and other modes of administration suitable for administering such dietary supplements.

In an alternative embodiment, the amount of LCPUFA and NORC in the composition is a function of an amount required to establish specified concentrations, or a desired range of concentrations, of LCPUFA and/or NORC in the blood serum of the animal. The specified concentrations, or desired ranges of LCPUFA and/or NORC in the blood serum may be calculated by determining the blood serum levels of animals fed the recommended amounts of LCPUFA and NORC specified above, as would be appreciated by one of skill in the art.

The sources of each of the LCPUFA can be any suitable source, synthetic or natural. Preferred sources of LCPUFA are natural sources of such fatty acids and include, without limitation, primrose, dark green vegetables such as spinach, algae and blue-green algae, such as spirulina, plant seeds and oils from plants such as flax, canola, soybean, walnut, pumpkin, safflower, sesame, wheat germ, sunflower, corn, and hemp, and fish, especially cold-water fish such as salmon, tuna, mackerel, herring, sea bass, striped bass, halibut, catfish, and sardines, as well as shark, shrimp, and clams, and the extracted oils of any one or more of the foregoing. The LCPUFA may also be synthetic, and as such may be produced according to any means suitable in the art, from any suitable starting material. It is to be understood the LCPUFA as used herein may comprise a blend of any one or more LCPUFA from any one or more sources, such as those exemplified above, whether natural or synthetic.

The sources of NORC include any suitable source, synthetic or natural. In various embodiments, the NORC comprises arginine. Presently preferred sources of arginine include, without limitation, animal and plant proteins. Non-limiting examples of plants considered rich in arginine content include legumes, such as soy, lupins, and carob, grains such as wheat and rice, and fruits, such as grapes. Seeds and nuts of plants such as cacao and peanut are also considered rich in arginine content. Non-limiting examples of animal proteins considered rich in arginine content include poultry and fish products. The NORC can also be synthetic, according to any means suitable in the art. Both LCPUFA and NORC, whether natural or synthetic, can be obtained directly or provided by a commercial source.

The compositions of the invention can optionally comprise substances such as minerals, vitamins, salts, condiments, colorants, and preservatives. Non-limiting examples of minerals that may be useful in such compositions include calcium, phosphorous, potassium, sodium, iron, chloride, boron, copper, zinc, magnesium, manganese, iodine, selenium and the like. Non-limiting examples of vitamins include such fat soluble vitamins as A, D, E, and K, as well as water-soluble vitamins including various B vitamins, as well as vitamin C, for example. Niacin, pantothenic acid, inulin, folic acid, biotin, amino acids, and the like may be useful to include in various embodiments.

The compositions of the invention can optionally comprise one or more substances that promote or sustain vascular health, or further enhance vascular integrity. Such substances include, without limitation, vinpocetine, coenzyme Q₁₀, phosphatidylserine, acetyl-L-carnitine, alpha-lipoic acids, extracts, such as that from Bilberry (Vaccinium myrtilis), or antioxidants, including tocopherols, tocotrienols, carotenoids such as alpha- and beta-carotene, lycopenes, luteins, astaxanthin, zeaxanthine, flavonoids such as flavanols, flavones, flavanones, flavan-3-ols (e.g. catechins), anthocyanidins, isoflavones (e.g. isoflavonoids), betacyanins, anthoxanthins, and the like.

In various embodiments, pet food or dietary supplement compositions of the invention can comprise, on a dry matter basis, from about 15% to about 50% crude protein, by weight. The crude protein material may comprise vegetable proteins such as soybean, cottonseed, and peanut, or animal proteins such as casein, albumin, and other animal proteins, such as from meat. Non-limiting examples of sources of animal protein useful herein include porcine, ovine, equine, avian (e.g. poultry), and species such as fish and sea animals, or any combination thereof.

The compositions may further comprise, on a dry matter basis, from about 5% to about 40% fat, by weight. The compositions may further comprise a source of carbohydrate. The compositions may comprise, on a dry matter basis, from about 15% to about 60% carbohydrate, by weight. Non-limiting examples of such carbohydrates include grains or cereals such as rice, corn, sorghum, alfalfa, barley, soybeans, canola, oats, wheat, and mixtures thereof. The compositions may also optionally comprise other materials such as dried whey and other dairy products and/or by-products.

The compositions may also comprise at least one fiber source. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide additional to the short chain oligofructose, mannanoligofructose, soy fiber, fiber from lupins, arabinogalactan, galactooligosaccharide, arabinoxylan, or mixtures thereof. Alternatively, the fiber source can be a fermentable fiber. Fermentable fiber has previously been described to provide a benefit to the immune system of a companion animal. Fermentable fiber or other compositions known to those of skill in the art which provide a prebiotic composition to enhance the growth of probiotic microorganisms within the intestine may also be incorporated into the composition to aid in the enhancement of the benefit provided by the present invention to the immune system of an animal. Additionally, probiotic microorganisms, such as Lactobacillus or Bifidobacterium species, for example, may be added to the composition.

In particular embodiments, the composition is a complete and nutritionally balanced pet food. In this context, the pet food may be a wet food, a dry food, or a food of intermediate moisture content, as would be recognized by those skilled in the art of pet food formulation and manufacturing. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15%, and therefore is presented, for example, as small biscuit-like kibbles. The compositions and dietary supplements may be specially formulated for specific animals based on their age or heath status. For example, formulation specific for puppies, or kittens, or formulations for animals as they get older, for example aging or senior animals are contemplated for use herein. In general, specialized formulations will comprise energy and nutritional requirements appropriate for animals at different stages of development or age, or at different risk of ischemic events.

Certain embodiments provided herein, for example for companion dogs and cats, are preferably used in combination with a complete and balanced food (for example, as described in National Research Council, 1985, Nutritional Requirements for Dogs, National Academy Press, Washington D.C., or Association of American Feed Control Officials, Official Publication 1996). That is, compositions comprising LCPUFA, or DHA according to certain aspects of this invention are preferably used with a high-quality commercial food. As used herein, “high-quality commercial food” refers to a diet manufactured to produce the digestibility of the key nutrients of 80% or more, as set forth in, for example, the recommendations of the National Research Council above for dogs. Similar high nutrient standards would be used for other animals.

The skilled artisan will understand how to determine the appropriate amount of LCPUFA and NORC to be added to a given composition. Such factors that may be taken into account include the type of composition (e.g., pet food composition, dietary supplement, or food product formulated for human consumption), the average consumption of specific types of compositions by different animals, and the manufacturing conditions under which the composition is prepared. Preferably, the concentrations of LCPUFA and NORC to be added to the composition are calculated on the basis of the energy and nutrient requirements of the animal. According to certain aspects of the invention, the LCPUFA and NORC can be added at any time during the manufacture and/or processing of the composition. This includes, without limitation, as part of the formulation of the pet food composition, dietary supplement, or food product formulated for human consumption, or as a coating applied to the pet food composition, dietary supplement, or food product formulated for human consumption.

The compositions can be made according to any method suitable in the art such as, for example, that described in Waltham Book of Dog and Cat Nutrition, Ed. A T B Edney, Chapter by A. Rainbird, entitled “A Balanced Diet” in pages 57 to 74, Pergamon Press Oxford.

Methods

Another aspect of the invention features methods for enhancing the vascular integrity in an animal comprising administering to the animal a composition comprising one or more LCPUFA and one or more NORC in an amount effective to enhance vascular integrity in the animal. Yet another aspect of the invention features prophylactic methods for reducing ischemia-induced injury to the brain of an animal comprising administering to the animal on a regular basis a composition comprising one or more LCPUFA and one or more NORC in an amount effective to reduce ischemia-induced injury to the brain in the event of an ischemic episode in brain the animal.

In particular embodiments of either of the two above-mentioned aspects of the invention, the composition is a pet food composition, a dietary supplement, or food product formulated for human consumption, as provided herein. In a further detailed embodiment, the LCPUFA is one or more of an (n-3) LCPUFA, including but not limited to EPA, DPA and DHA, and the NORC is one or more of L-Arg and derivatives thereof. Animals can include any domesticated or companion animals as described above, or can include humans. In certain embodiments, the animal is a companion animal such as a dog or cat. In another embodiment, the animal is a human.

The compositions can be administered to the animal by any of a variety of alternative routes of administration. Such routes include, without limitation, oral, intranasal, intravenous, intramuscular, intragastric, transpyloric, subcutaneous, rectal, and the like. Preferably, the compositions are administered orally. As used herein, the term “oral administration” or “orally administering” means that the animal ingests or a human is directed to feed, or does feed, the animal one or more of the inventive compositions described herein.

Wherein the human is directed to feed the composition, such direction may be that which instructs and/or informs the human that use of the composition may and/or will provide the referenced benefit, for example, the enhancement of vascular integrity in the animal or reduction of ischemia-induced injury to the brain in the event of an ischemic episode in the animal. Such direction may be oral direction (e.g., through oral instruction from, for example, a physician, veterinarian, or other health professional, or radio or television media (i.e., advertisement), or written direction (e.g., through written direction from, for example, a physician, veterinarian, or other health professional (e.g., prescriptions), sales professional or organization (e.g., through, for example, marketing brochures, pamphlets, or other instructive paraphernalia), written media (e.g., internet, electronic mail, or other computer-related media), and/or packaging associated with the composition (e.g., a label present on a container holding the composition).

Administration of the compositions is preferably provided on a regular basis. As used herein, a “regular basis” can be with any useful frequency, for example the compositions ca be provided on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the animal, or otherwise contacted with or admixed with daily feed or food or drinking water, or other beverage. When utilized as a daily feed or food, administration will be well known to those of ordinary skill.

Administration can also be carried out on a “regular basis”, for example, as part of a diet regimen in the animal. A diet regimen may comprise causing the regular ingestion by the animal of a composition comprising one or more LCPUFA and one or more NORC in an amount effective to enhance vascular integrity or to reduce ischemia-induced injury to the brain in the event of an ischemic episode in the animal. Regular ingestion can be once a day, or two, three, four, or more times per day, on a daily or weekly basis. Similarly, regular administration can be every other day or week, every third day or week, every fourth day or week, every fifth day or week, or every sixth day or week, and in such a dietary regimen, administration can be multiple times per day, for example where the composition is formulated as part of a food or feed, or admixed therewith, and the feeding is ad libitum. The goal of regular administration is to provide the animal with an effective dose of LCPUFA and NORC. In some embodiments, the regular administration preferably provides a daily dose of LCPUFA and NORC, as exemplified herein.

According to the methods of the invention, administration of the compositions comprising one or more LCPUFA and one or more NORC, including administration as part of a diet regimen, can span a period of time ranging from gestation through the entire life of the animal.

The following examples are provided to describe the invention in greater detail. They are intended to illustrate, not to limit, the invention.

### EXAMPLE 1

### Effect of 17β-estradiol or Dietary Supplementation with Arginine, Fish Oil or Combination on Brain Damage from Transient Cerebral Ischemia in Ovariectomized Rat Model

Chronic treatment with 17β-estradiol (E2) was compared with three test diets containing arginine, LCPUFA in the form of fish oil, or a combination thereof, respectively, for their effects on brain damage caused by transient cerebral ischemia in an ovariectomized rat model.

Methods:

Animals. Female Charles Rivers Sprague-Dawley rats (250 g, Wilmington, Mass.) were acclimatized to animal facilities three days prior to surgery with a 12 hour light-dark cycle. Bilateral ovariectomy was performed 2 weeks before diet feeding began. Four weeks after the start of diet feeding, transient middle cerebral artery (tMCA) occlusion under anesthesia was performed following intraperitoneal (i.p.) injection of ketamine (60 mg/kg) and xylazine (10 mg/kg).

Diet and Hormone Administration. Fourteen to 15 rats per group were randomly assigned to one of five treatment groups. These groups were as follows:

Group 1 Control diet (White Diet)

Group 2 Control diet+SILASTIC® estradiol implant for 1 week (White+E2)

Group 3 Diet I (Pink Diet)

Group 4 Diet II (Purple Diet)

Group 5 Diet III (Gray Diet)

The control diet was a standard rat diet containing 140 g/kg casein, 100 g/kg sucrose, 50 g/kg fiber, 155 g/kg dextrin, 466 g/kg corn starch, 35 g/kg standard salt mix, 40 g/kg soybean oil, 10 g/kg standard vitamin mix, 1.8 g/kg L-cystine and 2.5 g/kg choline chloride. All three test diets (Diets I, II and III) were supplemented with (1) additional B vitamins, including 45 mg/kg nicotinic acid, 35 mg/kg pantothenate, 24 mg/kg pyridoxine, 15 mg/kg thiamin, 9 mg/kg riboflavin 3 mg/kg folic acid, 0.8 mg/kg biotin and 0.225 mg/kg vitamin B12, and (2) an antioxidant cocktail comprising 500 mg/kg vitamin E, 150 mg/kg vitamin C, 100 mg/kg astaxanthin and 0.4 mg/kg selenium. Diet I further included 2% arginine (20 g/kg). Diet II further included 2% arginine and 2% menhaden fish oil (20 g/kg). Diet III further included 2% menhaden fish oil.

All diets were fed ad libitum to the rats for four weeks. Food changes occurred once per week. Also once per week, body weights and food intake were determined. One week before the induction of a tMCA occlusion, Group 2 rats were administered E2 at a dose of 4 mg/ml in a SILASTIC® pellet that was implanted subcutaneously. This procedure has been used to protect the brain from a variety of insults and produces physiologically relevant level of serum E2 (Simpkins et al., 1997; Jung et al., 2003). This was a positive control, since it has been shown previously that this dose and time of E2 treatment resulted in protection from the effects of tMCA occlusion (Simpkins, et al., 1997; Yang et al., 2004a, Yang, et al., 2004b).

Middle cerebral artery occlusion and samples preparation. Animals were anesthetized by an intraperitoneal injection of ketamine (60 mg/kg) and xylazine (10 mg/kg). tMCA occlusion was performed as previously described (Simpkins et al., 1997) with slight modification. Briefly, the left common carotid artery, external carotid artery and internal carotid artery were exposed through a midline cervical incision. A 3.0 mono-filament suture was introduced into the internal carotid artery lumen and gently advanced until resistance was felt. The surgical procedure was performed within 20 minutes, with minimal bleeding. The suture was kept in place for 60 minutes and then withdrawn to allow reperfusion. Rectal temperature was monitored and maintained between 36.5 and 37° C. with a heating pad throughout the procedure. At 24 hours after the onset of reperfusion, the animals were sacrificed and the brains were removed. The brains were then dissected coronally into 2 mm sections using a metallic brain matrix (ASI Instruments Inc.; Warren, Mich.) and stained by incubation in a 2% solution of 2,3,5-triphenyltetrazolium chloride (TTC) in physiological saline at 37° C., and then fixed in 10% formalin.

DNA fragmentation analysis with TdT-mediated dUTP Nick-End Labeling (TUNEL) MCA occlusion is a widely used focal ischemic stroke model (Bederson et al., 1986). This in vivo model for neuronal death can rapidly induce a synchronized apoptotic process in a large number of neurons and other cells (Li et al., 1997). The effects of transient ischemia on apoptosis were therefore examined by analyzing DNA fragmentation with the TUNEL assay. TUNEL staining was performed according to the modified manufacturer's instructions (Gavrieli et al., 1992). Formalin-fixed, paraffin-embedded tissue sections were deparaffinizated with xylene, rehydrated through graded ethanol treatment, and given a final wash in PBS. The sections were post-fixed in 4% paraformaldehyde for 20 minutes. Sections were then washed and treated for 15 minutes with 100/μg/ml proteinase K in PBS, equilibrated with equilibration buffer for 10 min, and then incubated with buffer containing TdT enzyme and FITC-labeled dUTP (Promega, Madison, Wis.) at 37° C. in a humidified chamber. The reaction was terminated by incubation in 2×SSC buffer for 15 min at room temperature. The sections were then mounted with anti-fade reagents containing DAPI (Molecular Probes, Eugene, Oreg.). Positive control sections were immersed in DNase I solution for 10 min at room temperature before equilibration in TdT buffer. The sections were observed under a fluorescent microscope with appropriate excitation/emission filter pairs.

Some animals were eliminated from the study during the protocol. The number of animals quantified for lesion volume ranged from 12 to 15 per group.

Statistical methods. Results were analyzed with one-way analysis of variance (ANOVA) using Prism software(Graphpad Inc; San Diego, Calif.). The significance of differences among groups was determined by one-way ANOVA followed by Tukey's multiple comparison tests. All values were expressed as mean±SEM.

Results:

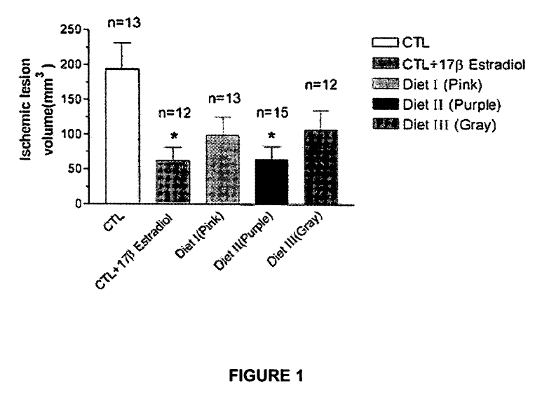

Stroke Volume. All four experimental conditions (estradiol and Diets I, II and III) reduced infarct size (FIG. 1). Estradiol treatment (Group 2) reduced infarct volume by 68%, a value typical of an estrogen protection from stroke damage (Simpkins, et al., 1997; Fan et al., 2003; Yang et al., 2004a; Yang et al., 2004b). Similarly, Diet II reduced infarct volume significantly, by 67%. The two other diets tested also reduced mean infarct volumes, but the data were too variable for the values to be statistically significant.

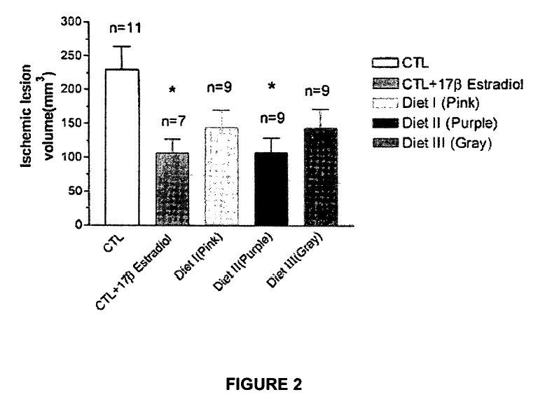

Inasmuch as each group had rats with no observable lesions (value of 0 in our calculations of lesion volume), the data were assessed after these values were omitted, to determine the extent to which the 0 values contributed to the group differences. As shown in FIG. 2, the same two groups were significantly lower than the control diet group with this modification in the data. Thus, the differences among groups were driven by the animal in which lesions were observed as well as by the number of animals without observable lesions. The protective effects of both estrogen treatment and diets were exerted primarily on the cortex, an area of the brain called the penumbra, as it is believed to be “rescueable” in experimental stroke. This is in contrast to the basal ganglia, called the core of the infarct, which is not readily saved by any treatment tested to date in experimental stroke.

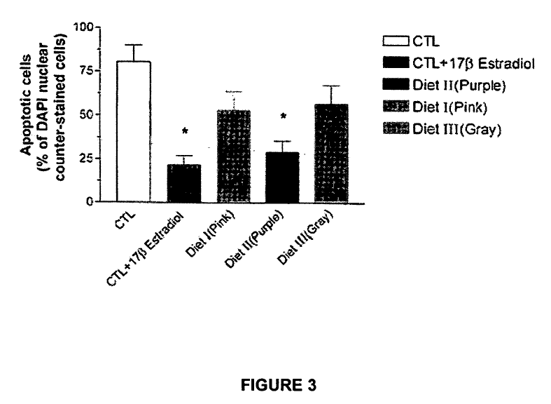

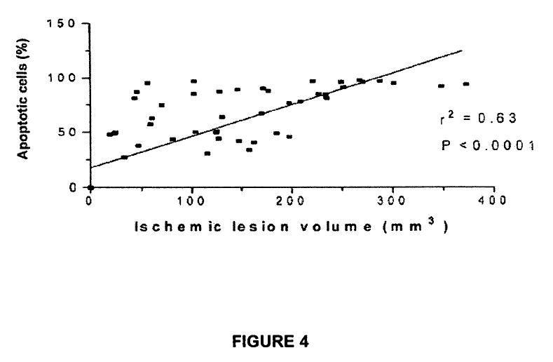

Apoptosis. Assessment of apoptosis using TUNEL staining was conducted to determine the consequences of estradiol treatment and the three test diets on the apoptotic response to experimental ischemia. Three brain regions for TUNEL staining were assessed in all animals that completed the study. TUNEL was assessed in the core of the cerebral cortex, the penumbra of the cerebral cortex and the core of the subcortex, based upon our assessment of damaged brain regions using TTC staining (Wen et al., 2004). The number of TUNEL positive cells was normalized to the total number of cells in the field using DAPI nuclear counter staining. Sections from all three brain regions were counted and their average cell counts for each animal were used to generate the group means. Cell counts were conducted in randomly selected microscopic fields (320 μm square sections) in slices from the maximum extent of the brain infarct, as determined by TTC staining. TUNEL staining was extensive in all three brain regions in the control animals. When a comparison of all five treatment groups was done, the TUNEL staining in each brain correlated with the results found using TTC staining to quantify lesion volume (FIG. 3). That is, there was a substantial reduction in TUNEL staining in both the E2-treated animals as well as animals fed Diet II. Animals fed Diets I and III were intermediate between controls and the Diet II animals. This correlation between TTC staining and TUNEL staining is depicted in FIG. 4. The characterization of the effects of hormones and diets on TUNEL staining indicates that a substantial portion of the protection by E2 and Diet II from cell death is mediated by an inhibition of apoptosis.

Conclusions:

All three test diets reduced mean infarct volume as well as TUNEL-positive cell counts and Diet II reduced these parameters to the level seen with a known neuroprotectant, estradiol.

The present invention is not limited to the embodiments described and exemplified above, but is capable of variation and modification within the scope and spirit of the appended claims.

What is claimed:

1. A composition comprising one or more long chain polyunsaturated fatty acids (LCPUFA) and one or more nitric oxide releasing compounds (NORC) in an amount effective for the enhancement of vascular integrity in an animal.

2. The composition of claim 1 wherein the composition is a pet food composition, dietary supplement, or a food product formulated for human consumption.

3. The composition of claim 2 that is a dietary supplement providing about 0.5 to 10 g/day of each of LCPUFA and NORC.

4. The composition of claim 1 wherein the LCPUFA include at least one of arachidonic acid, eicosapentaenoic acid, docosapentaenoic acid, and docosahexaenoic acid.

5. The composition of claim 1 wherein the NORC include at least one of L-arginine and derivatives thereof.

6. The composition of claim 1, comprising at least about 0.1% to about 13% LCPUFA, and at least about 0.1% to about 12% arginine, by weight.

7. The composition of claim 6, comprising about 1% to about 3% LCPUFA, by weight.

8. The composition of claim 6 comprising about 1% to about 3% NORC, by weight.

9. The composition of claim 1 further comprising at least one antioxidant.

10. The composition of claim 9 that provides about 0.1 mg to about 5 g of antioxidant.

11. The composition of claim 1 further comprising one or more B-vitamins in an amount that about 1 to 5 times a recommended daily allowance (RDA).

12. The composition of claim 1, wherein the animal is a companion animal.

13. The composition of claim 9, wherein the companion animal is a dog or cat.

14. The composition of claim 1, wherein the animal is a human.

15. A method for reducing ischemia-induced brain injury in an animal, comprising administering to the animal on a regular basis a composition comprising one or more LCPUFA and one or more NORC in an amount effective to reduce ischemia-induced brain injury in the event of an ischemic episode in the brain of the animal.

16. The method of claim 15 wherein the composition is a pet food composition, a dietary supplement, or a food product formulated for human consumption.

17. The method of claim 15 wherein the LCPUFA include at least one of arachidonic acid, eicosapentaenoic acid, docosapentaenoic acid, and docosahexaenoic acid.

18. The method of claim 15 wherein the NORC include at least one of L-arginine and derivatives thereof.

19. The method of claim 15, wherein the composition comprises at least about 0.1% to about 13% LCPUFA and at least about 0.1% to about 12% L-arginine, by weight.

20. The method of claim 15, wherein the animal is a companion animal.

21. The method of claim 20, wherein the companion animal is a cat or dog.

22. The method of claim 15, wherein the animal is a human.

23. The method of claim 15 wherein the regular basis comprises administration at least once daily.

24. A method for enhancing the vascular integrity in an animal, comprising administering to the animal on a regular basis a composition comprising one or more LCPUFA and one or more NORC in an amount effective to enhance the vascular integrity of the animal.

25. The method of claim 24 wherein the composition is a pet food composition, a dietary supplement, or a food product formulated for human consumption.

26. The method of claim 24 wherein the LCPUFA include at least one of arachidonic acid, linoleic acid, alpha-linolenic acid, eicosapentaenoic acid, docosapentaenoic acid, and docosahexaenoic acid.

27. The method of claim 24 wherein the NORC include at least one of L-arginine and derivatives thereof.

28. The method of claim 24, wherein the composition comprises at least about 0.1% to about 13% LCPUFA and at least about 0.1% to about 12% L-arginine, by weight.

29. The method of claim 24, wherein the animal is a companion animal.

30. The method of claim 29, wherein the companion animal is a cat or dog.

31. The method of claim 24, wherein the animal is a human.

32. The method of claim 24, wherein enhancing the vascular integrity of the animal results in a reduction in brain damage in the event of an ischemic episode in the brain of the animal.

33. The method of claim 24 wherein the regular basis comprises administration at least once daily.
